(19) United States
(12) Patent Application Publication
(10) Pub. No.: US 2011/0129479 A1
(43) Pub. Date: Jun. 2, 2011

(54) IMMUNOGEN SELECTION DIRECTED IN IMMUNOGLOBULIN PACKAGES IN PLASMA AND COLOSTRUM AND METHOD OF MAKING AND USING SAME

(76) Inventor: MONTE B. TOBIN, VENICE, FL (US)

(21) Appl. No.: 12/957,839

(22) Filed: Dec. 1, 2010

Related U.S. Application Data
(60) Provisional application No. 61/283,288, filed on Dec. 2, 2009.

(51) Int. Cl. A61K 39/395; A61K 39/40; A61K 39/42; A61P 37/04; A61P 31/04; A61P 31/12; A61P 31/16; A61P 31/14; A61P 31/20; A61P 31/10

(52) U.S. Cl. 424/140.1

(57) ABSTRACT

A method for the selection and production of group specific immunoglobulins to bind to specific microbes or their toxins is provided. The immunoglobulins are produced in animals and collected either in the plasma or colostrums of the animals after being challenged with a series of selected immunogens. The selected immunoglobulins are packaged into products against specific groups of microbes or their toxins. These packages are delivered to animals including humans. These products could be used as passive protectants. The packages could be developed into products to protect animals or humans against diseases until vaccines can be effectively administered.

### RELATED APPLICATIONS

This application claims the benefit of U.S. Provisional Patent Application Ser. No. 61/283,288, filed 2 Dec. 2009, and entitled “Immunogen Selection Directed In Immunoglobulin Packages in Plasma and Colostrums and Method of Making and Using Same.”

### FIELD OF THE INVENTION

This invention is directed to the production of immunoglobulins in colostrums or plasma for the diagnosis or treatment of specific conditions.

### BACKGROUND OF THE INVENTION

Immunoglobulins, or antibodies, are proteins that are found in blood or other bodily fluids of animals and are used by the immune system to identify and neutralize foreign objects, such as bacteria and viruses.

Immunoglobulins can come in different varieties known as isotypes or classes. In placental mammals there are five immunoglobulins isotypes known as IgA, IgD, IgE, IgG and IgM. Each class differs in their biological properties, functional locations and ability to deal with different immunogens.

The most common immunoglobulins are IgA and IgG. IgA is found in mucous secretions such as tears, saliva, colostrum and in the blood. IgA can survive in harsh environments such as the respiratory and digestive tracts. IgG is the most abundant immunoglobulin and has two antigen binding sites. IgG is located in the blood and tissue liquids. IgG can bind to many types of pathogens such as viruses, bacteria and fungi.

The structures and functions of immunoglobulins are fairly uniform through out the animal kingdom including bovine equine immunoglobulins. The antigenic differences and biological activities of the different classes of immunoglobulins correlate with the physico-chemical differences of the groups. The groups vary in concentrations depending on which body fluid they are found in. The serum or plasma have higher concentrations of IgG immunoglobulins while the exocrine secretions will have higher IgA concentrations. Colostrum usually has higher IgG concentrations but recent studies would show that IgM may be more efficient.

Specific immunoglobulins can be produced by injecting an immunogen into a mammal, such as a mouse, rat or rabbit for small quantities of antibody, or goat, sheep, or horse for large quantities of antibody.

Bovine immunoglobulins are in general like any other animal immunoglobulins. The immunoglobulins can be found in the serum, plasma or other body fluids such as colostrums. Bovine immunoglobulins are divided into the classic five groupings: IgG, IgM, IgA, IgD and IgE. Differences do exist between breeds of cattle and how they are challenged to make immunoglobulins. These differences can be overcome by using a variety of breeds of cattle to product specific immunoglobulins or even specific antibodies to selected antigens. Quantity of immunoglobulins can be adjusted with the immunization schedule.

All groups of immunoglobulins are quite stable in aqueous solutions. They can withstand high salt concentrations for purification purposes and low pH's for further purification. The phase of purity of the molecule plays a role in the molecules stability.

Bovine immunoglobulins are some of the most stable proteins and can be active into the GI Tract. Bovine immunoglobulins can be heated to 60° C. for short periods and withstand digestive enzymes in the stomach and small intestine. The IgG group has 2 to 4% carbohydrate hexose content needed for some biological activities. The same Bovine Immunoglobulin classes are found in serum or plasma, milk and colostrums. They only differ in concentrations. Using specialized bovine colostrums or immunoglobulins in animal studies, bovine immunoglobulins have been shown to have a high capacity for neutralizing of bacterial toxins and viruses and high effectiveness in treating severe diarrhea.

Bovine colostrums alone may contain from 8% to 16% Igg's. It is known that colostrum can be enhanced by immunizing the cow 30 to 45 days before calving. The colostrums obtain the immunoglobulin faction concentrations from the serum.

The production of immunoglobulins in colostrums or plasma for the diagnosis or treatment of specific conditions has been known. The production of immunoglobulins in colostrums and plasma using specific vaccines or delivery system has been known. The prior art teaches methods for immunizing animals and collecting milk, colostrums, and/or plasma from the immunized animals. The prior art further teaches processing the collected colostrum, milk or plasma to create general products.

It is known to vaccinate cows or other animals with specific immunogens to get specific antibodies in either colostrums or serum. It has also been suggested to provide these to other animals, including humans to treat or prevent diseases. It is also known to provide these antibodies to increase performance rather than treat diseases.

Various methods of processing the immunoglobulins are used. For example, some methods remove the fat from the colostrums, or sterilize the colostrums.

Various methods of distributing the immunoglobulins are used. For example, immunoglobulins can be distributed to an animal via a spray or ingestion. The immunoglobulins can be processed to take many forms including liquid, dry powder or gel. The immunoglobulins can be mixed with other formulations.

A number of reports have indicated that in both animal and human trials colostrum contains specific immunoglobulins which are effective against both enteropathogenic and enterotoxigenic organisms. Since most infectious agents enter the body proper via the epithelial surfaces of either the upper respiratory, digestive or genitor-urinary tract, mucosal protectants in the form of immunoglobulins could be helpful. The function of the immunoglobulins is to bind to invading microbes and to activate specific actions that help rid the body of disease causing microbes. They aid in the functions of cell killing, inflammation and prevention of microbe attachment including viruses, bacteria, fungi and their toxins.

The benefits of using bovine immunoglobulins in animal feed is well established, as they have been shown to improve efficiency of dietary protein utilization, reduce pathogen attachment and replication in the intestinal tract, helps maintain gut barrier function, reduces local inflammation of the small intestine and less mucosal damage in the intestinal tract and less tight junction protein damage to allow the intestinal tract cells to function properly.

Equine immunoglobulins are very similar to bovine in terms of concentrations in the serum versus the colostrums. The standard Igg's are of the IgG, IgA IgM classes. There are five subclasses of IgG referred to as Ga, Gb, Gc, G(B) and G(T). The IgG(T) is rich in carbohydrate and is in high concentrations of body fluids. The T stands for the reactivity to tetanus. The highest concentrations of IgG are found in both the serum and colostrums as in bovine. The colostrum does differ in the number and subtypes. Horses have almost 10 times the concentration of Igg's in the colostrums than found in milk. Foals have a gestation period of 340 days. The fetus can responded at day 79 to stimulus of the immune system. Newborn foals may have some IgM and IgG with a little IgG(T) in the serum at birth. Plasma cells to do not appear until day 240 of gestation. Newborn foals have a selective intestine absorption of the immunoglobulins. IgG and IgM are taken in but SIgA is left in the intestine.

### SUMMARY OF THE INVENTION

The method of the present invention may include selecting at least one target immunogen, selecting a producing animal, inoculating the at least one producing animal with the at least one target immunogen, allowing a period of time sufficient to permit the production of immunoglobulin to the at least one target immunogen in the at least one producing animal, harvesting at least one fluid containing immunoglobulins from the at least one producing animal, processing the at least one fluid to create at least one immunoglobulin package, and administering the at least one immunoglobulin package to at least one target animal.

The selecting a producing animal step of the method may include selecting a healthy animal, profiling the animal's IGG's, and comparing the animal's IGG's to a predetermined minimum.

The processing the at least one fluid step of the method may include analyzing the fluid for the targeted IGG's and manufacturing the fluid to create an immunoglobulin package delivery system.

The at least one producing animal of the method is selected from the group consisting of cows, horses, sheep, camels, goats, rabbits and llamas.

The at least one target immunogen of the method may be capable of decreasing an animal's ability to utilize feed by causing respiratory disease.

The at least one target immunogen of the method may be chosen from the group consisting of P. Multicoda, M. haemolytica, H. somnua and H. suis.

The at least one target immunogen of the method may be chosen from the group consisting of Mycoplasma pleuropneunmoniae, M. hypopneumoniae and M. bovis.

The target immunogen of the method may be capable of causing respiratory illness in humans.

of the method t least one of the target immunogens of the method may be from the class of respiratory viruses such as influenza (H1N1, H3N2, H5N1).

The target immunogen of the method may be capable of causing respiratory complex in humans.

At least one of the target immunogens of the method may be a respiratory virus.

At least one of the target immunogens of the method may be selected from the group consisting of Bovine respiratory syncytial virus, Bovine viral diarrhea, adenoviruses, bovine parainfluenza 3, and infection bovine rhinotracheitis virus.

At least one target immunogen of the method may be chosen from the group consisting of Eastern Encephalomyelitis, Western Encephalomyelitis, Venezuelan Encephalomyelitis, Influenza A1, Influenza A2, EHV1 and EHV4.

The target animal of the method may be a feed animal.

The administering of the at least one immunoglobulin package to the target animal of the method may inhibit the ability of the immunogen to adhere to the respiratory tract of the target animal and reduce the ability of the immunogen to multiple.

The immunogen of the method may be PRRS.

The immunoglobulin package of the method may be mixed with a carrier material.

The carrier material of the method may be chosen from the group consisting of soybean oil, molasses, whey, rice hulls, soy bean hulls, pellets, distilled dried grains, boluses, tablets, PBS buffer, vitamin E solution and beet pulp.

The delivery system of the method may be chosen from the group consisting of powder, capsules, spray, gel, liquid, salve, ointment, or cream.

The fluid containing immunoglobulins of the method may be plasma.

The fluid containing immunoglobulins of the method may be colostrums.

The method may include the producing animal being a pregnant animal in its last trimester of pregnancy.

The method may include both plasma and colostrums being harvested from the producing animal.

The method may include at least one target immunogen being a Gram Positive microbe.

The method may further include at least one target immunogen being chosen from the group consisting of Streptococcus, Staphylococcus, Bacillus, Listeria, Clostridium botulium, tetanus and Enterococcus

The method may include at least one target immunogen being a Gram Negative microbe.

The method may include at least one target immunogen being chosen from the group consisting of E. coli, Salmonella, Haemophilus, Helicobacter, Campylobacter and Yersinia.

The method may include at least one target immunogen and the producing animal being chosen from the same geographical area.

The method may include the producing animal being inoculated with more than one target immunogen.

The method may include at least one target immunogen being an immunogen capable of causing respiratory illness in companion animals.

The method may include at least one target immunogen being an immunogen capable of causing respiratory illness in high value nonfood animals.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

The present invention relates to a method for the selection and production of group specific immunoglobulins to bind to specific microbes or their toxins. The immunoglobulins are not specific to a given strain of microbe. The immunoglobulins are produced by injecting an immunogen or groups of immunogens to generate a broad spectrum of immunoglobulins in animals such as bovine or equine. These protocols may include injecting pregnant bovine or equine during the last part of the pregnancy. The immunoglobulins may be collected from the plasma and/or the colostrums and early milk. The plasma will provide a broad spectrum of polyclonal immunoglobulins while the colostrums will be limited to a majority of IgG type immunoglobulins with some IgA and IgM immunoglobulins.

Selection of the animals and profiling their immunoglobulin patterns before the immunogen challenge will allow for the development of unique groups of immunoglobulins not found in the normal population. The collected immunoglobulins can then be mixed as needed to manufacture a series of products containing a broad spectrum of cross-reacting immunoglobulins. The uniqueness of the packages allow for products that could be used to benefit most animals including bovine, equine, canine, etc and humans.

Preferably, the selected microbes or their toxins from a targeted region and selected animals are from the same targeted region to utilize the unique immunoglobulin pattern from the region.

Animals can be selected from various geographic areas that have been exposed to regional environmental microbes. These animals may have a wide range of animal vaccinations or be raised on the open grassland that would add to the unique environmental immunoglobulin profile. The selected animals preferably not only have the unique microbe profile for the area but could be boostered with selected immunogens to produce an increased broad spectrum of immunoglobulins. This method of selecting animals is unique and would produce a full spectrum of cross-reacting immunoglobulin packages that are not found in the normal population or in any other current products. The animals that are selected must be healthy and tested to make sure that they are free of common pathogens or disease.

These unique mixes or packages could be engineered as needed. Microbes and their toxins found in a geographic area or region could be selected to produce specific immunogens that could be used to produce specific products throughout the world. Those found in numerous areas could be created in one place and shipped throughout the world. Regional profiles that could be identified could be reproduced on one farm and shared within the region. Cross-reacting reagents would be useful throughout the area.

The selected packages of immunoglobulins can be delivered to the various animal groups or humans in a variety of ways. The packages can be added to standard rations and fed orally. They can be left in a liquid condition and sprayed on to feed or sprayed in the nostrils. For animals or humans, the packages can be placed into capsules and taken orally as supplements. There are unlimited means of delivering bioactive materials in the same formulations.

The present invention addresses the problem of preventing microbes from being shed or transmitted from animal to animal, animal to human, or human to human. The immunoglobulin packages could be aimed at stopping the organisms at the source by binding them at the source so they can not be spread easily. The immunoglobulin packages may be manufactured into products that can be administered immediately upon need. Emergency medical personnel could carry these products during potential epidemics. People could take these products before they go into contaminated areas. Animals could be mass treated while giving vaccines time to work. The same could be done with humans. These products preferably have long shelf lives. They may be in the form of capsules, nasal sprays, gel swabs, liquids, oral mists, nasal gels, tropical salves/ointments/creams and/or throat mists. In some cases such as Bird Flu, there could be more than one form of delivery of the product to prevent the spreading of the organisms.

The present invention provides a rapid system that will produce immunogens that may be used to immunize cattle. The cattle respond to the specific immunogens and produce antibodies to these structures. The structures have been selected to cross-react with given groups of microbes such as Pasteurella, Hemophilus, Mycoplasma, BVD viruses, Influenza viruses such as H1N1, H3N2 and H5N2. Any group of viruses, bacteria, toxins, or parasites could be the starting immunogens.

Groups of Gram positive organisms such as Tetanus, Clostridium, Botulism, Listeria, Corynebacterium, Staphylococcus, Streptococcus, Bacillus and others may be selected as immunogen targets. This may include pathogens such as methicillin resistant Staphylococcus aureus (MRSA). Propionibacterium acnes may be used for making a salve product for acnes.

Groups of Gram negative organisms such as Escherichia, Helicobacter, Klebsiella, Salmonella, Yersinia, Shigella, Vibrio and others may be selected as immunogens for targeted packages. This may include parasites such as Coccoidia and Cryptospridium. These immunoglobulin packages may be mixed with rations to help with GI tract problems.

Selected targets for equine use may be Tetanus, Streptococcus, botulium, E. coli, Ehrlichia, or others to produce unique packages. Groups for respiratory problems may be selected from influenza A1 and A2, Rhinopneumontis (EHV-1 and EHV-4), Eastern and Western Encephalomyelitis, West Nile Virus, Venezuelan Encephalomyelitits, Rhodococcus or others for immunoglobulin targets. Products may be made with a broad spectrum of these immunoglobulins.

The present invention allows for the development of specific immunoglobulins that bind and cross-react with groups specific immunogens. The antibodies are preferably collected from the cattle using any method known in the art. The immunoglobulins are then preferably past through a 0.2 micron filter and made into products. This process removes any potential contaminating microbes and thus can not be transferred from animal to animal or animal to human as happens with other processed products. The products can be used at various levels to block the spreading or shedding of the microbes. The following are all processes for making products containing immunoglobulin packages.

Broadly stated, This method includes steps of identifying the target microbe or their toxin and making of immunogens; selecting animal to inoculate to product immunoglobulins; inoculating the animal and waiting a predetermined amount of time for immunoglobulins to be produced; collecting, analyzing and storing fluid containing immunoglobulins; and manufacture immunoglobulin containing fluids into a final delivery system.

The step of identifying the microbe or their toxin and making of immunogen starts with identifying a need. Once the need is identified, the correct microbe or their toxin is selected. This grouping could be a unique microbe to a given geographic region or just a select group of microbes. Immunogens are then made using a specific protocol. The broadest set of immunogens should be used to booster the animal's immunoglobulin level. Once selected, immunogens are made to cross-react with the specific group of microbes or toxins. The immunogens are produced using a special process for stabilizing the immunogens to give the broadest stimulation.

The present invention is includes the step of selecting the problem causing microbe or their toxin. This could be a specific regional concern or geographic in nature. Once the group microbe is identified then immunogens have to be obtained to provide the broadest coverage for the region. These immunogens may be obtained from commercially available vaccines or through the use of specifically isolated microbes and made into an autogenous vaccine for the region. The key is to pick as many immunogens as possible to stimulate a package containing a broad spectrum of immunoglobulins. Not only does one want standard cell wall immunogens but any unique immunogen that can be developed. The cultures of microorganisms may be obtained from commercial sources such as the American Type Culture Collection (ATCC) or isolated from the environment. The cultures may be used to isolate immunogens. Autogenous vaccines can be made from the unique microbes isolated from the farm or region. The cultures can be prepared as prepared immunogens and may be injected intra-muscularly, but preferably injected subcutaneously. If specific commercial vaccines are available and they represent a broad spectrum of immunogens, they can be part of the vaccine program. This works well if a series of vaccines are available from more than one company or the company has a number of strains in the vaccines.

For most bovine products, bovine animals are selected from either a targeted region or country or from regions within countries to maintain the environmental footprints of the geographic area. This provides a broad spectrum background. The Jersey strain gives the highest immunoglobulin concentration and the animals of choice. The animals are evaluated as to their vaccination history and current health status. The animals are tested for any disease microbes that they may be carrying. Many times the older cows or culled cows have the broadest ranged profile. The animals have to be found to be in a state of health before they are considered further for the production. Samples of the animal's plasma are tested to provide the immunoglobulin profile before immunogens are selected. Once selected the animals are injected with the immunogens using a specific schedule unique to the specific microbe or toxin.

If colostrum is needed for calves or human products, then the healthy pregnant cows would be the animal of choice. Selected animals will be vaccinated during the last trimester of the pregnancy. This allows for increase IgG's in the first milkings. Unless vaccination is done properly, the IgM levels and IgA levels will not increase in the colostrums. If this is the case, the plasma of the animal will have to be utilized. The colostrum is collected using the first four milkings after the birth. Starting one week later, plasma is collected from the cow for the next three weeks.

For most equine products, equine animals are selected from either the targeted region or country or from regions within countries to maintain the environmental footprints of the geographic area. This provides a broad spectrum background. The animals are evaluated as to their vaccination history and current health status. The animals have to be found to be in a state of health before they are considered further for the production. Samples of the animal's plasma are tested to provide the immunoglobulin profile before immunogens are selected. Once selected the animals are injected with the immunogens using a specific schedule unique to the specific microbe or toxin.

If colostrums is needed for foals, then the healthy pregnant horse would be the animal of choice. Selected animals will be vaccinated during the last trimester of the pregnancy. This allows for increase IgG's in the first milkings. The colostrum is collected using the first four milkings after the birth. Starting one week later, plasma is collected from the horse for the next three weeks.

For other animals such as goats or sheep similar protocols as done for the bovine or equine materials are utilized.

All mammals provide similar types of protection which allow for an immediate immune response in their very young offspring until they too acquire the ability to make the immunoglobulins for themselves. More specifically called passive immunity protection, this defense mechanism is passed to the young of mammals through the placenta, the mother's colostrum, mother's milk, plasma or through all ways. The young of cattle and horses, however, receive their passive immunity protection through the store of immunoglobulins placed in the colostrums from the serum during the development from the embryonic stage. If the cows or dams are vaccinated during the pregnancy they will concentrate immunoglobulins from the serum in the colostrums. Once immunized, the cows deposit the IgG type immunoglobulins in the colostrum while depositing lesser amounts IgM and IgA immunoglobulins. The mother will also deposit immunoglobulins against immunogens she has been in contact in the local environment in the colostrums. Horses do the same thing but tend to have more IgA in the colostrums than found in cows colostrums. Furthermore, the large quantities of immunoglobulins which are placed in colostrums during the last trimester are much more exclusively those specific for the immunogens to which the mother has most recently been exposed to and challenged by. This all results in the immunoglobulins of these animals being a most idea source for large quantities of economically produced highly specific and stable immunoglobulins found in the plasma. While the invention is illustrated by the use of bovine or equine to produce specific immunoglobulins, other animals including goats, sheep, llamas, camels or combination thereof, may be used.

Specifically, groups are obtained of cows typically Jersey, Holsteins, Guernsey crosses or other breeds or horses, typically Belgium or other cross breeds suited to high volume immunoglobulin production and ease of handling which have been in the region or geographic area so they have had time to develop immunoglobulins to a broad spectrum of microbes found in the environment, on a schedule predetermined by the amount and timing of final product desired resulting in a steady continuous production stream. It is best if there are vaccination records to aid in the review to see if the animals meet the minimum requirements. The animal is examined for any health issues and blood samples are taken for analyze to make sure they are not carrying any standard herd diseases. The older the animal such as culled cows or older steers could also be used. A sample of blood is taken for a compete profiling of the immunoglobulins already being produced by the animal.

After a suitable period of isolation and acclimatization of about two to four weeks, each animal will enter into an inoculation program using rehydrated proprietary preparations of specific antigens (immunogens) to which an immunoglobulin is desired. Depending upon how broad the immunogen pools are a set schedule is set up for each group of immunogens. This could be 4-6 injections over a four week period. In approximately four to five weeks, the average animal will produce copious amounts of the desired specific immunoglobulin in a readily usable and stable form. The animals may be reinnoculated with the targeted immunogen throughout the harvest period to maintain the high immunoglobulin level. Since animals vary in their immune status, at least two animals are arranged per group.

If colostrums products are needed then animals are selected as given above except the cows need to be in the third trimester. The animals are only vaccinated with killed bacterins. All Vaccinations are given in the neck area and only S.C. injections.

Once the vaccination series is complete, the animals are rested for one to two weeks. Blood samples are taken to determine if enough immunoglobulin has been produced by the animals. The Jersey strains do the best and have the highest Igg levels of all strains of bovine. If the animals are ready, plasma is collected using a plasmapheresis unit. This allows for the fluid to be removed but not the cells. This causes less stress on the animal. These animals can have plasma harvested on a weekly basis if needed. Mature cows can give 9.5 to 10 liters of plasma per week. Horses can be harvested every two to three weeks with the amount varying with the size of the animal. Once the plasma is collected, samples are taken for analysis. Standard test procedures are used, such as ELISA, agglutination, or the like are used to the monitor the immunoglobulin activity. Total protein and Total Igg's concentration are also monitored. The batch is made from a combination of harvest samples from the same animal over at least a three week period and may be mixed with the other animals in the group if all specifications are met.

If colostrum is collected, it is collected after the birth of the calf or foal. Starting at the day the calf is born, the vaccinated cow will be milked using a specific sequence. The colostrums will be collected for the first four milkings to make a batch. The colostrums are collected and pooled. All colostrums is sampled and tested for Total Protein, Total Igg and specific tests such as ELISA, agglutination, or the like are used to monitor the immunoglobulin activity. The samples of the mixture are analyzed to make sure the specific immunoglobulins are in the colostrums sample. Those colostrums that meet the minimum level are then frozen until further processed. Colostrums from animals in the same group can be pooled if all specifications are met. For the equine or other animals, a similar procedure can be followed.

These animals are then milked for two weeks followed by harvest of plasma once a week for three weeks. The plasma is preferably collected using a selected plasmapheresis method. The plasma is analyzed to make sure the specific immunoglobulins are in the plasma sample. If the material meets the minimum required level, it is frozen until needed. The plasma can be pooled within the groups if all samples meet the specifications. The cow will be plasmaphoresed for the next two weeks or until the levels of specific immunoglobulins drop. For other bovine, the animals are plasmaphoresed until they get too old to be sampled or until the levels of the specific immunoglobulins drop below the minimum levels. For equine or other animals, a similar procedure can be followed.

The materials can then be manufactured into their final delivery system. The delivery systems to deliver these colostrums/plasma packages may take many forms. For example, the products can be delivered in a powder form, packaged into capsules and delivered like a pill or left in liquid to form a spray or gel. Salves, ointments or creams can be made for topical applications.

The materials can be manufactured into separate packages or mixed. The materials that meet the minimum requirements can be mixed to make large batches. The typical batch is then blended with batches from groups of plasma at other average production levels resulting in abundant standardized active ingredients. The plasma materials can be mixed to give a broad spectrum package of immunoglobulins that can be mixed with animal feed rations and feed to healthy animals such as bovine, equine, canine, etc. Human products could be manufactured that would have similar mixtures. The products can be liquid or powdered. Colostrum can be mixed with the plasma when it is needed to add specific IgG immunoglobulins to give a broad spectrum polyclonal package.

The immunoglobulin package material may be stored and shipped on carrier materials such as soy bean hulls, pellets, with soy oil, boluses and/or tablets. Dependent on the needs and specifications of the formulator and the final customer, the final immunoglobulin products may include some type of innocuous additive, such as dried whey or soy hulls, molasses, soy or rice husks or the like for formulation with feed ration. This method provides for the first time, an economical, safe and effective means for producing immunoglobulin packages for use in beef cattle and dairy herds, swine, chickens, turkeys, companion animals, horses, high value nonfood animals, zoological animals and humans.

Products containing colostrums can be made by mixing pools of colostrums from different immunogen groups with pooled plasma from still other groups of animals vaccinated with other immunogens. This material can be dried and delivered in a powder formulation. These products would have broad range and could be given safely to both animals as well as humans.

It is also contemplated that the plasma and colostrum will be collected and material from the groups will be mixed in the proper concentration with a carrier mixture such as molasses, soy oil, PBS buffer and Vitamin E solution. This solution is optimized so it can be sprayed, squirted, injected intra-nasally, gelled, or used on top feed and in lick tubs. The protective material may be sprayed over the animals in the pens or feedlots during the feeding period usually once in the morning and once in the evening. The number of sprayings is determined from testing. Since the material is non-toxic, it is given as needed and as much as needed for a given pen. The preferred method is by dried powder or liquid given orally. Animal products can be given as direct intra-nasal injection with a spray using dose per nostril or a combination of direct nasal spray plus top feed, lick tank, squirted, etc.

The immunoglobulin package contents incorporating the cross-reacting immunoglobulins specific to the targeted immunogens may be administered to the animals or humans by distributing the packaged material directly or introducing immunoglobulin material into the air. The packaged material may be introduced into the nasal pharyngeal area of the animal by direct injection with a syringe or sprayed. The packaged material may be administered with a spray doser directly or intranasal inoculation. Aerosol mixtures may be made and administered as a mist over the heads and nostrils of the animals. Another alternative is to mix the material with a carrier and administer as “top dressing” on feed. Special needs may be met by adding the packaged material to water and letting the animals or humans drink the solution. The active packages may be added to bulk licks or feed baskets for delivery. Gel-like mixtures may be made using common animal feed mixtures and pouring into “lick tanks” (feed additive liquid bulk tank). Other delivery systems may be adapted for delivery of the active material to the respiratory tract. The products may be used at various levels to block the spreading or shedding of the microbes.

Any microorganism which colonizes the nasal pharyngeal region of the respiratory or GI tract of its host must possess the capability of sticking or adhering to the surface of the mucus membranes in order to multiply. The respiratory pneumonia complex organisms such as Pasteurella multocida, M. haemolytica, Haemophilus somnus, Influenza viruses and Mycoplasma bacteria are no exception to the rule. Other microorganisms from the fungi and parasite groups could be included in organisms that may cause respiratory problems in animals or humans. The immunoglobulin packages of this invention strongly interferes with adherence and on a cumulative basis, thereby prevents the specific targeted microorganism from colonizing, and multiplying and moving down the respiratory tract and infecting the lower tract including the lungs or the GI tract. Through the vehicle of a simple nasal injection, spray, lick tank, the product essentially supplies the host with specific immunoglobulin packages designed not to cure any disease in the animal but merely to dislodge any resident microorganism and to prevent the attachment of any newly introduced microorganism in the upper respiratory tract. The immunoglobulin packages has no direct effect on the host itself, is all natural, leaves absolutely no undesirable residue in the animals, and thus has no effect whatsoever on the ultimate food products. In addition, since the microorganism is prevented from multiplying, it will over time (for example 21-30 days) disappear through natural degradation from mucus of the animal, eliminating the significant potential source of contamination in the feedlot. Properly managed, the risk of cross contaminating other animals throughout the feedlot is lowered and essentially eliminated. Similar applications could be developed for companion animals, zoological animals or nonfood animals or humans. They too have respiratory and GI tract problems. The bovine immunoglobulins are used as passive protectants to aid in getting the animals into a healthy state. These products help bridge the gap between the stress period and when the animal can build an active immunity to the stressor. These products that can be mixed in lick tank formulas and have shown active for at least 7 days. If the mixture has ingredients such as molasses the molecules will last even longer. The product can be made as a liquid or a dried powder as needed for delivery. Immunoglobulin packages can be formulated for receiving cattle with a starting program, for the first week to four weeks, depending on the intake level of the cattle. The packages can be formulated to add to lick tank formulations. The packages can be delivered by a spray into the nostrils of the animals.

The products are all natural preparation that contains specific immunoglobulins to the targeted immunogens. These immunoglobulins when attached to the outer surface cell wall, receptors, pilii or pilated structures and capsule, or viral capsid will not allow the organism to attach to the mucous membranes. By spraying the material, the mist will coat the nasopharynx and prevent the bacteria, viruses or other microorganisms from being spread in water droplets. The mist will also coat the feed and water in the area, again blocking the ability of the organisms to spread from animal to animal. The calves or foals will be healthier from birth and have a strong start to building a healthy immune system. This will give the animal a chance to meet its full growth potential. An active immune system could lead to improve daily gain, improve performance, improve feed efficiency, and reduce costs. Similar examples can be obtained in companion animals or humans.

The unique immunoglobulin packages for specific geographic regions throughout the world will make special products that not only will recognize a binding attachment but may also act as a trigger for their many types of defensive activities. Specific immunoglobulin binding and coating combined with the very likely mobilization of many other cellular defense systems, therefore, quickly culminating in the chemical inactivation and ultimately the destruction of the targeted microorganism. Colostrum and plasma mixture may actually stimulate the active immunity of the animal through the small intestine and pylorus batches. It is apparent that many modifications and variations of this invention as hereinbefore set forth may be made without departing from the spirit and scope thereof. The specific embodiments described are given by way of example only.

The invention is further illustrated by the following examples:

### Example 1

### Selection of Specific Targets for Calves

The first example for a model is that of E. coli which causes scours in newborn calves. The strains of E. coli should be selected strains that may be found within the region. Again a good model for a region would be the State of Wisconsin where a number of dairy herds can be found. There are a number of commercial vaccines available that could be selected as long a broad spectrum of immunogens can be given to stimulate a good immune response. A series of vaccines such as Scour Immune, E. coli Bacterin B. Pili Shield, Guardian E. coli and Bovine E. coli Shield could be purchased. The selected animals would be vaccinated over a 35 day period. The suggested schedule would be days 0, 7, 14, 21, and 28. They would be boostered with a general vaccine such as J-Vac on day 35. The animals would be tested to make sure the immunoglobulins have been boosted to give a good base line level. All animals may be harvested for plasma as long as the levels of Igg stay up. If the levels drop, the animals can be boostered again or culled and new animals started.

### Example 2

### Selection of Specific Targets in Different Geographic Regions

As a model E. coli can be used but the country of India poses a problem for a range of commercial vaccines. One could purchase an ATCC vulture of E. coli that could be found in the area or a recent isolate from the region could be used as a stock E. coli. Since a broad spectrum of immunogens are needed to booster the spectrum of immunoglobulins the immunogens can be modified to make autogeneous local vaccines following the methods put forth by Herzberg et al (1972, Degree of Immunity Induced by Killed Vaccines to Experimental Salmonellosis in Mice, Infect. Immun. 5(1):83-90) and Nash (1972, Development of a Pasteurella Bacterin for Bighorn Sheep (Ovis Canadensis Canadensis) PHD Thesis, Colorado State University, Ft. Collins, Colo. 102 pp.). The basic steps are as follows:

a. Preparation of Stock Culture

The American Type Culture Collection E. coli was used as the model bacterium. The ATCC Method for rehydration of the stock was followed. Transfer to 5 ml of TSB sterile broth. Incubate overnight (approximately 18 hrs) at 37° C. See nice turbid growth. Use this as Stock as needed. Streak on Sorbitol-MacConkey Agar (Difco) for verification of colony production.

b. Preparation of H antigens for Immunogens

The H antigens were selected for development into an immunogen. Certain conditions are used to maintain the optimum growth of the H antigen during culturing to give add concentrations for the prep. Veal Infusion Agar (VIA) and Veal Infusion Broth (VIB, Becton Dickinson) is preferred for H7 antigen production. From Stock TSB inoculate VIB. Incubate at 22-24° C. or room temperature for 18 hrs. This stimulates flagella development on the bacteria. Good growth after 22 hours. Harvest after 4 days. Combine flasks by washing off the agar surface with Dulbecco's PBS solution (pH 7.3-7.4). Collect in tubes. Check density using spectrophotometer enumeration and McFarland nephelometer standards. Approximately 3×10/12/ml in Stock. Check motility with Motility agar slant (Northeast Laboratory Services). Dilute stock concentration to approximately 1×109 per l in PBS. Stir for 1 hr at RT. Removes the Flagella from the outside of the bacteria. Determine dry weight approximately 14.7 mg/ml. Stock for H immunogen. Dilute to 1 mg/ml in PBS. Heat preparation for 30 minutes at 60-70° C. This helps keep contamination down to minimum. Inoculate thioglycollate broth to check for growth. Inoculate animals with immunogen.

c. Preparation of O Antigen for Immunogens

Brain Heart Infusion (BHI, acumedia) is used to stimulate the O antigens on the bacterium. From Stock TSB, inoculate BHI Broth. Incubate at 37° C. for 18 hrs. This stimulates somatic antigen development on the bacteria. While stirring slowly, incubate flasks at 37° C. Good growth after 22 hours. Combine flasks. Harvest using centrifugation and sterile saline (0.9%). Collect in tubes. Check density using spectrophotometer enumeration and McFarland nephelometer standards. Dilute to approximately 1×109 per ml. Add 4% sodium deoxycholate (Difco) solutions as 1:1 ratio with culture in 0.90 sterile saline (Herzberg et al, 1972). Centrifuge to remove whole cells. Use supernatant as stock for O antigen. Determine dry weight. Dilute in sterile PBS, pH 7.4 to 1 mg/ml for O immunogen.

d. Preparation of WC Antigen for Immunogens

Tryptic Soy Broth (TSB, Northeast laboratory Services) plus Yeast Extract (BBL) for SD Antigen Production. From Stock TSB, inoculate TSB plus Yeast Extract 0.6% Broth. Incubate at 37° C. for 18 hrs. This stimulates somatic and other surface antigens to development on the bacteria. Inoculate flasks with TSB with Yeast Extract Broth. Combine flasks. Harvest using centrifugation. Collect in tubes. Resuspend in sterile PBS, pH 7.4. Check density using spectrophotometer enumeration and McFarland nephelometer standards. Dry weight approximately 19.7 mg/ml. Dilute to approximately 2×109 per ml or 2 mg/ml dry weight. Add 0.4% formaldehyde solution in PBS as a 1:1 ratio with culture. Stir for approximately 18 hours at RT (22-24° C.) to fix cells. Inoculate thioglycollate broth to check for growth and check pH of preparation (pH 7-7.4). Use supernatant as stock for WC Immunogen. Dilute stock in PBS, pH 7.4 to 1 mg/ml for WC immunogen.

e. Preparation of A antigen for Immunogen

The Minca Medium is used for A Antigen Production. It is a standard medium for stimulating the pilii and related adhering antigens. From Stock TSB, inoculate Minca Medium Broth (Inf. Immun., February 1977, 676-678). Inoculate flasks with Minca Medium Broth. While stirring slowly, incubate at 37° C. Good growth after 18 hrs. Combine flasks. Harvest using centrifugation. Collect in tubes. Resuspend pellet in PBS. Stir with stir bag for 1 hour at 22-24° C. (RT). This removes the flagella. Collect in tubes. Resuspend pellet in PBS and 0.01% Tween 20™.

Transfer to Waring Blender in cold (4° C.). Low speed for 30 minutes. Check density using spectrophotometic enumeration and McFarland nephelometer standards. Centrifuge to remove whole cells. Use supernatant as stock for A immunogen. Heat at 60° C. for 40 min. to inactivate if needed. Add gentamycin at 50 mg/ml as preservative. Inoculate thioglycollate broth to check for growth. Determine dry weight. Approximate 10.6 mg/ml. Dilute with PBS, pH 7.4 to 1 mg/ml for A Immunogen.

Using each of the immunogen preparation inject the animals on day 0, 7, 14, and 21. Use 2 ml of the selected immunogen. The schedule should be WC immunogen on day 0, O immunogen on day 7, H immunogen on day 14 and A immunogen on day 21. Using equal amounts of the immunogens to make up a booster injection. This is given on day 35. Blood samples are taken from the animals as needed to monitor the levels of immunoglobulins to E. coli. The plasma can be collected as needed.

### Example 3

### Selection of Specific Targets for Equine

The first example for a model is that of Equine respiratory problems such as Strangles in newborn foals and mares. The Equine strains should be selected that may be found within the region. There are a number of commercial vaccines available that could be selected as long a broad spectrum of immunogens can be given to stimulate a good immune response. A series of vaccines such as Pinnacle, StrepVax II, Streptococcus equi and S. zooepidermidis could be purchased. The selected animals would be vaccinated over a 35 day period. The suggested schedule would be days 0, 7, 14, 21, and 28. They would be boostered with a general vaccine such as StrepVax II on day 35. The animals would be tested to make sure the immunoglobulins have been boosted to give a good base line level. All animals may be harvested for plasma as long as the levels of Igg stay up. If the levels drop, the animals can be boostered again or culled and new animals started.

### Example 4

### Selection of Specific Targets for Equine

The first example for a model is that of other Equine respiratory problems in newborn foals and mares. The Equine strains should be selected that may be found within the region. There are a number of commercial vaccines available that could be selected as long a broad spectrum of immunogens can be given to stimulate a good immune response. A series of vaccines such as Eastern, Western, and Venezuelan Encephalimyelitits, Influenza, Rhodococcus could be purchased. The selected animals would be vaccinated over a 35 day period. The suggested schedule would be days 0, 7, 14, 21, and 28. They would be boostered with a general vaccine on day 35. The animals would be tested to make sure the immunoglobulins have been boosted to give a good base line level. All animals may be harvested for plasma as long as the levels of Igg stay up. If the levels drop, the animals can be boostered again or culled and new animals started.

### Example 5

### Preparation of ELISA Plates Used for Monitoring Immunoglobulins in Plasma, Colostrums and Feed

IMMUNOGEN ELISA: Ninety-six well assay plate (flat bottom Costarâ) were coated using 100 ml/ml with various concentrations of individual Immunogen or combination of same: The immunogens were diluted to various concentration of 10 mg-200 mg/ml) in carbonate buffer, pH 9.6. Plates were incubated between 22-37° C. for up to 18 hrs. The wells were aspirated to prevent cross-contamination. The plates were blocked with 390 ml/well of 0.5% BSA and incubated at 37° C. for up to 18 hr. Plates are rinsed 1× with wash buffer containing Tween™ 20. Two hundred microliters per well of diluted sample are added to wells in duplicate wells. Incubated at 37° C. for one hour. Goat anti-bovine Igg conjugate with Horseradish peroxidase (Kirkegard and Perry laboratories; 1:1000-1:3000) was added. After 1 hr incubation, the substrate (TMB, KPL) was added according to manufacturer's instructions and the reaction is stopped after 10 minutes with 0.1M phosphoric acid. Optical densities of the wells were determined in Dynatech ELISA Reader at 450 nm and the information was recorded for further data analysis.

### Example 6

### Sample results from Respiratory ELISA Plates

The following is a table with examples of results for ELISA's using immunogens from the selected calf respiratory microbes. These examples would be Pasteurella (PM), Haemophilus (HS), Mycoplasma (Ma), and bovine viruses. The plasma samples are mixed into the formulation and then tested with the selected ELISA plates:

TABLE 6a
Results of Samples Diluted and Tested with
Select Immunogen Coated ELISA

| Immunogen | Dilution | O.D |
|---|---|---|
| PM | 500 | 0.532 |
| PM | 2500 | 0.123 |
| Ma | 500 | 0.456 |
| Ma | 2500 | 0.125 |
| HS | 500 | 0.378 |
| HS | 2500 | 0.128 |
| CVa | 500 | 0.598 |
| CVa | 2500 | 0.155 |

### Example 7

### Sample results from Selected ELISA Plates

The following is a table with examples of results for ELISA's using immunogens from the selected immunogens in liquid batches analyzed after the total protein and total Igg assays. These examples would be Pasteurella and Haemophilus immunogens used to coat the plates. The liquid samples are mixed into the formulation and then tested with the selected ELISA plates:

TABLE 7a
Sample results of ELISA Assays using Selected
Immunogens

| Batch: Liquid | | | | |
|---|---|---|---|---|
| Pasteurella Immunogen Signal/Noise | | | | |
| Haemophilus Immunogen Signal/Noise | | | | |
| Batch #1 | 0.367 | 2.32 | 0.211 | 2.68 |
| Batch #2 | 0.288 | 2.91 | 0.275 | 2.93 |
| Batch #3 | 0.372 | 2.97 | 0.238 | 2.91 |

### Example 8

### Selection of Bovine animals for Plasma Production

The strain of bovine may vary with needs and uses. Any bovine animal may be immunized including dairy cattle, cows, steers or even bulls. Culled dairy cows are preferred because they have been trained to stand in holders for longer periods of time. The common strains of bovine are the preferred and are usually selected for the concentration of immunoglobulins they can generate and ease of handling. Jersey, Guernsey and Holstein cows of average dairy size usually meet these criteria. The short-horned (polled) animals work the best as to gentle handling. Animals can be selected from culled cows on a farm or at sale barns. All animals must have a clean record of good health. Animals that are older (2-3 years or more) seem to have the best profile for immunoglobulin patterns. All animals are tested for BVD, Johnes and Mycoplasma. This is done are certified labs using direct counts and PCR testing. Immunoglobulin profiles using the Immunogen ELISA's are done on the individual serum samples. Once the animals meet the initial specifications they are divided into groups. For Example as in Example #1, the animals have good concentration of immunoglobulins to E. coli they can be placed in the E. coli group. At least two animals need to be in each group but as many as needed can be added to the group. They are then vaccinated according the schedule given in Example #1 or if in a different geographic area Example #2. The plasma is then harvested as needed. These animals can be utilized until no longer needed. Depending upon the schedule, the animals may be needed to be boostered on a quarterly basis as needed.

### Example 9

### Selection of Pregnant Cows for Colostrum Production

When colostrum is needed for a product, cows must be selected for the vaccination period. The strains of animals are not important but the Jersey, Holstein, or combinations of strains are the preferred animals. The animals need to be in good health. The ideal is to have the animal in the last trimester of the pregnancy. Since harm could be done to the developing calf only kill bacterins or vaccines should be used on these animals. Once the animal meets these criteria, then serum samples are taken to make sure the animals are not carrying any unknown diseases. The samples are monitored using both direct culturing and PCR to rule out BVD, Johne's or Mycoplasma. Again, if the animals check out, they are assigned to a given group for vaccination. Again a minimum of two animals are needed for each grouping. If one uses the Example #2 were the animals are in a geographically area like India, the animals can be vaccinated with the different WC, O,A,H or combination immunogens. This schedule has to be done within the last days before the birth of the animal. The cow is vaccinated and left to rest until birth during the last 10 to 15 days. This vaccination schedule should not only booster the IgG levels in the milk but if done right should add IgM and IgA. These are minor but significant components in the immunoglobulin pool that will show up in the colostrum. Once the animal gives birth to the calf, the colostrums is collected over the next four milkings. This gives the highest levels of immunoglobulins. After that time, the colostrums will turn to milk and have about 1/10th the level of Igg's in the fluid milk. These cows will be milked for two weeks and then plasma is harvested once a week for three weeks or until the levels of immunoglobulins drop below the specification level. The colostrums and plasma are then run through the QC tests. If the samples all meet specifications they are mixed to make products.

### Example 10

### Selection of Equine animals for Plasma Production

The strain of equine may vary with needs and uses. Any equine animal may be immunized including draft horses, quarter horses, mares or stallions. The stock draft horses that weigh 1800 to 2200 lbs work the best. The Belgium strain has the right temperament but almost any well trained horse would work. Animal in the 8 to 16 year range have the best immunoglobulin profiles. The draft horses are preferred because they have been trained to stand in holders for longer periods of time. The common mixed strains of equine are the preferred and are usually selected for the concentration of immunoglobulins they can generate and ease of handling. Belgium horses usually meet these criteria. Animals can be selected on a farm or at sale barns. All animals must have a clean record of good health. Immunization records are very helpful for review. Animals that are older (8 years or more) seem to have the best profile for immunoglobulin patterns. All animals are tested for Equine infectious anemia (EIA), Piroplasmosis (Babesia equi {Theileria equi} and Babesis caballi), Dourine (Trypanosoma equiperdum), Glanders (Burkholeria mallei), Brucellosis, and Influenza. This is done by approved laboratories certified using direct counts and PCR testing. Immunoglobulin profiles using the Immunogen ELISA's are done on the individual serum samples. Once the animals meet the initial specifications they are divided into groups. For example Equine influenza, if the animals have good concentration of immunoglobulins to Equine Influenza they can be placed in the equine influenza group. At least two animals need to be in each group but as many as needed can be added to the group. They are then vaccinated according the schedule given in Example #1 or if in a different geographic area Example #2. The plasma is then harvested as needed. The plasma is traceable back to the date and location of collection. These animals can be utilized until no longer needed. Depending upon the schedule, the animals may be needed to be boostered on a quarterly basis as needed.

### Example 11

### Selection of Pregnant Horses for Colostrum Production

When colostrum is needed for a product, horses must be selected for the vaccination period. The strains of animals are not important but the Belgium or combinations of strains are the preferred animals. The animals need to be in good health. The ideal is to have the animal in the last trimester of the pregnancy. Since harm could be done to the developing foal only kill bacterins or vaccines should be used on these animals. Once the animal meets these criteria, then serum samples are taken to make sure the animals are not carrying any unknown diseases. The samples are monitored using both direct culturing and PCR to rule out Equine Influenza or EIA. Again, if the animals check out, they are assigned to a given group for vaccination. Again a minimum of two animals are needed for each grouping. For example if colostrums is needed for foals with high immunoglobulin levels to Equine Influenza were the animals are in a geographically area like Kentucky, the animals can be vaccinated with the different WC, O,A,H or combination immunogens. This schedule has to be done within the last 45 days before the birth of the animal. The cow is vaccinated and left to rest until birth during the last 10 to 15 days. This vaccination schedule should not only booster the IgG levels in the milk but if done right should add IgM and IgA. This are minor but significant components in the immunoglobulin pool that will show up in the colostrum. Once the animal gives birth to the calf, the colostrums is collected over the next four milkings. This gives the highest levels of immunoglobulins. Since 25% of foals fail to absorb sufficient quantities of immunoglobulins, the higher the level in the colostrums of IgG and IgM the better for the animal, After that time, the colostrums will turn to milk and have about 1/10th the level of Igg's in the fluid milk. These horses will be left to recover from the birth and then plasma is harvested once every three weeks for three collections or until the levels of immunoglobulins drop below the specification level. The colostrums and plasma are then run through the QC tests. If the samples all meet specifications they are mixed to make products.

### Example 12

### Preparation of Colostrum Based Products

Once the colostrum and plasma has been tested and passed from QC testing the products can be made. For example, a broad spectrum product can be made my mixing specific colostrums/plasma mixes from five different groupings. This could cover E. coli, Salmonella, Clostridium, Rotavirus and Coronavirus groupings. The formulation would be equal volumes of Colostrum and Plasma with 20 or more of dextrose or sucrose. This is mixed for 30 minutes to make sure it is homogenous. The mixture is then dried in a lypholizer using a standard freeze dry cycle. The powdered product is then tested for BVD, Johne's and Mycoplasma along with total plate counts. The product should be negative for all pathogens tested and less than 1000 cfu for the total plate counts. The protocol for collection of colostrums is done under aseptic methods. The plasma is collected with a plasmapheresis machine using plasma filtration that leaves the plasma free of microbes. When the powder has been tested, the results were negative for all pathogens and on 3 cfu/gm of product. This product can be mixed with rations or milk and given to animals.

### Example 13

### Preparation of Plasma for Products

Once the plasma has been tested and passed from QC testing the products can be made. For example, a broad spectrum products contain packages of immunoglobulins can be made my mixing specific plasma mixes from five or more different groupings. This could cover E. coli, Salmonella, Clostridium, Rotavirus and Coronavirus groupings. The plasma batches are sampled and tested for total protein and total Igg concentrations. The formulation would be equal volumes of Plasma groups based on the total Igg's. with whey, 80 or more of dextrose or sucrose, 1% soy oil and 24% PBS, pH 7.4. Potassium sorbate is added as a preservative at 0.50. This is mixed for 15 minutes to make sure it is homogenous. The mixture can be bottled aseptically or dried in a hot air dryer using a standard dry cycle. The liquid or powdered product is then tested for BVD, Johne's and Mycoplasma along with total plate counts. The product should be negative for all pathogens tested and less than 1000 cfu for the total plate counts. The plasma is collected with a plasmapheresis machine using plasma filtration that leaves the plasma free of microbes. When the powder has been tested, the results were negative for all pathogens and on approximately 30 cfu/gm of product. The liquid products were all negative for the same organisms. This product can be mixed with rations or milk and given to animals.

### Example 14

### Immunization of Cattle with Groups of Immunogens

Selected 10 head of cattle from sale barns. Most of the animals were culled cows. The animals were Holstein or mixtures and were injected with the commercial immunogens. The animals were divided two to a group. Four or five injections were given one week apart for 28 days. A varied of commercial products were used for each grouping to get a broad spectrum of reactions. The animals were injected SC along the neck. All vaccines were killed or non-living. The groupings included E. coli, salmonellae, Clostridiae, Rotavirus and Coronavirus immunogens. A serum sample was collected two weeks after the last initial injection. If boosters were needed, a 2 ml dose of mixed vaccines was given in each booster (approximately very six months). Within four weeks, all cattle produced excellent immunoglobulin packages in the serum. ELISA assays are used to monitor the specific groupings. Sample readings are as follows: EILSA readings averaged 1.00 OD for 1:10,000 dilution and 0.265 OD for 1:50,000.

### Example 15

### Examples of Immunogens for Enteric or GI Tract

A wide range of groups could be chosen for making immunoglobulin packages. The following is a list for enteric or GI tract conditions in Humans or animals.

### Enteric: GI Tract

Enterococcus E. coli

### Endotoxin Pseudomonas

### Enterovirus

Clostridium perfringens: Toxin Type A,B,C,D or E
Clostridium difficile Rotavirus

### Coronavirus Giardia

### Cryptospiridium Toxoplasma

### Salmonella Shigella

### Vibrio Actinomyces

Campylobacter Eubacterium: obesity
Helicobacter: ulcers

### Example 16

### Examples of Immunogens for Respiratory tract: Spray

A wide range of groups could be chosen for making immunoglobulin packages. The following is a list for respiratory tract conditions in Humans or animals.

### Respiratory Tract: Sprays

### Haemophilus Rhinoviruses

### Adenovirus Influenza: H1, H3, and H5

### Mycoplasma Pseudomonas

### Aeromonas Bordetella

### Example 17

### Examples of Immunogens for Skin Injuries: Salves or Creams

A wide range of groups could be chosen for making immunoglobulin packages. The following is a list for skin injuries as conditions in Humans or animals.

### Skin/Injuries: Salves/Ointments/Creams

### Neisseria Streptococcus

### Staphylococcus Chlamydia

Corynebacterium Propionibacterium: acne

### Mycobacterium Candidia

To list a few among others: etc.

### Example 18

### Preparation of Stock Immunoglobulin Packages

Selected plasma batches were combined from five to ten immunogen groups to be used to produce production batches of immunoglobulin packages. The plasma batches give the broadest range of immunoglobulins when more than one animal supplies the plasma and when the plasma was collected over a period of time. The best batches are mixtures of three or more collection dates. The use of one batch on only one day will not allow for reproducibility in the future production batches. The plasma is sampled and tested before adding to the mixture. All samples must be tested for BVD, Johne's and Mycoplasma plus doing total plate counts. The plasma is mixed as 50 to 560 of the product. Whey or other proteins as added at 8% weight followed by 10% sugar and 1% soy oil or vegetable oil. This is mixed with 24% PBS, pH 7.4. The potassium sorbate is dissolved in PBS and mixed at 0.50. The mixture is blended gently with a mixer for 15 minutes. The batch is sampled and tested for total protein and total Igg's. The sample should meet the minimum specification of 12.5 mg of Igg/ml. The mixture is then filtered thorough a series of filters. The filters start at 20-40 microns then to a 10 micron to 5 micron to a 0.45 micron and ending with a 0.2 micron filter. The product is then aseptically bottled in a clean room. Bottles are sealed and labeled. Samples are taken at the beginning, middle and end of the bottling run for analysis. Total protein must be spec of 12.5 mg/ml and all samples negative for pathogens.

### Example 19

### Preparation of Packages for Lick Tanks

The manufacturing process for the lick tank product in the liquid form is very simple and straightforward. The immunoglobulin packages are selected based on need. If the problem has to do with enteric problems a series of plasma's are selected to cover groups such as E. coli, Salmonella, Clostridium, Rotavirus, and maybe Cryptospriidium. These are mixed in predetermined amounts for the region and added to a mixture of protein whey, soy oil, PBS and a vitamin/mineral mixture. They are mixed, filtered and aseptically bottled. The bottles are labeled with a set distribution cup so just the right amount of material is delivered for each lick tank. In one trial, one lick tank was placed near the cattle in a pen of one hundred 600-pound steers. The cattle in the test feedlot were very interested in this material. They visit the lick tank several times a day. Consumption was about 5-10 ml/head/day. It is anticipated that per head consumption would be somewhat higher if more lick tanks were placed in the opened pen.

### Example 20

### Development of Feed Mix

Products can be developed to block the microbes from going from the feed into the animal at the source. One of the key preparations can be used for Feed Mix. Specific immunoglobulin packages are produced from animals immunized with selected enteric immunogens such as Salmonella, E. coli and Clostridium in equal amounts for a total of 7-9 L. The plasma material is added to 2 L of PBS, pH 7.4, whey and 4 L of distilled water. This is mixed well and preservatives such as potassium sorbate and sodium citrate are added to prevent microbial growth and extend shelf-life. A vitamin/mineral mix is added to the mixture. The total amount is 18 L. The mixture is stirred to get a homogenous solution. The mixture is then filtered. The material is bottled, is cooled and stored at 4° C. until used. This material is poured on top of the feed as needed. It usually is distributed once every 7 days for a total of three applications.

### Example 21

### Development of Material for Aerosol or Spray

One of the key preparations can be used for Aerosol or spray. Specific immunoglobulin packages are made from plasma collected from cows immunized with immunogens from respiratory microbe groups such as Pasteurella, Haemophilus, Mycoplasma, Bovine viruses and others such as Adenovirus. The plasma batches are mixed in equal amounts for a total of 10 L. The filtered plasma material is added to 6 L of PBS, pH 7.4 and 4000 gm protein whey. This is mixed well and preservatives such as food grade soy oil, potassium sorbate and sodium citrate are added to prevent microbial growth and extend shelf-life. The total amount is 18 L. The mixture is stirred to get a homogenous solution. The mixture is then filtered and aseptically bottled. The material is cooled and stored at 4° C. until used. This material is sprayed directly over the heads of the animals to form an aerosol. Workers can carry these loaded guns out on the range or in the feedlot pens and deliver directly to the cattle as needed. The material can be sprayed directly on the nose of the individual animals as needed. This makes for a very versatile means of application out on the range. It usually is distributed once every 7 days for a total of three applications or as needed.

### Example 22

### Products using Capsules

The immunoglobulin packages can be stored as powders materials and deposited into capsules. Our process allows for the drying of the product and placing it into capsules. This is a convenient means of delivery of the product. A wide range of packages could be made into product that can be made to block the microbes from going into the intestinal tract of humans or animals. They can be stored until needed. Microbes such as E. coli or Salmonella that cause diarrhea in humans could be prevented if the person took a capsule or two before they came in contact with the contaminated food or water. Rahimi et al, 2007 recently published a paper using the same type of concept. They showed a reduction of Salmonella in broilers and in the feed when they added specific developed immunoglobulins to the microbes. By adding immunoglobulins to the GI tract, the Salmonella or E. coli or whatever packages of immunoglobulins you need can be placed into the capsule. This could be used to prevent the transfer of various microbes such as Clostridium, E. coli and their toxins, Streptococcus, Campylobacter, Listeria, etc. Cross-reacting immunoglobulin packages could be developed to address all these microbes.

### Example 23

### Products using Nasal Spray

Examples of products using Compact Nasal Spray Products: It is important to prevent the shedding of microbes from one animal to another or one human to another. This not only threatens the herd or population but also creates the potential to cross-contaminate food products made from the animal. This could be used for any number of microbes that can be shed by feed animals or humans.

Model #1: A good model would be Influenza. This microbe can be shed from the animal without anyone knowing about it. It can be transferred to humans. Worse it can be transferred from human to human. Bird flu is the current problem. Problems have occurred in the current developed of new vaccines for the H5 antigens found in the current outbreaks among birds. We propose to develop immunogens to standard strains of group A Influenza including H1N1, H3N2 and H5H2. These antibody preparations will be mix and developed into a spray. The spray bottle can be carried by the person and administered to the nostril as needed.

Since these products are non-toxin they can be given immediately upon emergency. If a person gets, the flu they can use it to prevent the shedding and spreading of the disease. Large batches of the same product could be given to bird population using these new power sprayers. A whole broad spectrum of immunoglobulin packages could be developed and delivered this way. It appears that if the right immunoglobulins are sprayed the shedding of the organisms can not only be lowered but can lowered to below threshold levels or levels detected using standard laboratory methods of culturing of the organisms. This reduces the potential to cross-contamination.

Model #2: A good model to show the blocking of shedding would be nasal spray for BVDV immunogloblins. These antibodies could be sprayed into the nostrils of the cattle as needed. The products are non-toxic so they could be administered as needed. Veterinarians could identify the PI calves and administer the product in the nostrils as needed. This could be done with the power doser's using the air tanks. It makes it a portable in the field.

Model #3: A good model would be the foot and mouth disease. Currently, there is a new vaccine being developed. This product would use killed antigens and Immunogens would be made from these antigens. The products produced would be a series of specific immunoglobulins that would be made into a spray product The product could be made and stockpile for future use. The antibodies could be sprayed into the nostrils of the cattle. It would form a bridge of time for the vaccines to work and protect the cattle. The added benefit would be that the animals would not be shedding during this time or would they be as susceptible to the virus because the antibodies in the mucus would block the entry of the virus into the animal. This is critical to the spread of this virus.

### Example 24

### Process using Gel Swabs

This method uses gel swabs to deliver the immunoglobulin packages. This method of delivery would work will for both animals and humans. Products could be made for a variety of microbes. It would work for both animals and humans. The gel swab can be contained in a plastic case until needed. Humans could carry it with them and use it on the nostrils as needed. A Veterinarian could carry it to prevent transmission among animals as needed.

### Example 25

### Process using Liquid product

This method uses Liquid Cup Products to deliver the immunoglobulin packages. This is a simple method to produce the product but leaving it as a liquid. For humans, a small cup could be used to deliver the product as needed. Any GI tract problem could be addressed. Selected microbes such as scour causing organisms such as E. coli, Salmonella, rotaviruses, coronaviruses, coccidia, Clostridium, PRRS, Circovirus, etc can be addressed with this technology. Blends of materials can be made into feed rations and feed the animals at a young age. A similar group of products could be developed for Human needs.

### Example 27

### Process using Oral Mist product

This method uses Oral Mist Products to deliver the immunoglobulin packages. This delivery method could be used for cold or sore throat problems. The products could be made against Streptococcus, Rhinoviruses, Enteroviruses, or other common human problems. This could be misted right into the mouth and direct delivery.

### Example 28

### Process using Nasal Gel Product

This method uses Nasal Gel Products to deliver the immunoglobulin packages.

This delivery method has the advantage of delivering a sticky gel that will mix with the mucus and form a barrier to block the entry of the microbe into the body. It could be used for a variety of unique pathogens.

### Example 29

### Process using Throat Spray product

This method uses Throat Spray Products to deliver the immunoglobulin packages. This would be similar to the Nasal Spray but a unique delivery system. It allows for a different storage unit and the spray tip allows for direct delivery to where the product is needed. It would be mainly used for Human products.

### Example 30

### Process using Topical Salves or Creams for Skin Product

This method uses topical salve for skin problems products to deliver the immunoglobulin packages. The immunoglobulin packages are mixed with the salve or cream and used as topical skin products.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method of producing an immunoglobulin package comprising:
selecting at least one target immunogen;
selecting at least one producing animal, wherein the at least one producing animal and at least one target immunogen are chosen from the same geographical area;
inoculating the at least one producing animal with the at least one target immunogen;
waiting a predetermined period of time sufficient to permit the production of immunoglobulin to the at least one target immunogen in the at least one producing animal
harvesting at least one fluid containing immunoglobulins from the at least one producing animal; and
processing the at least one fluid to create at least one immunoglobulin package.

2. The method of claim 1 wherein the selecting at least one producing animal step further comprises:
selecting a healthy animal;
profiling the healthy animal's immunoglobulin concentration;
comparing the healthy animal's immunoglobulin concentration to a predetermined minimum immunoglobulin concentration.

3. The method of claim 1 wherein the processing step further comprises:
analyzing the at least one fluid for the targeted immunoglobulin; and
manufacturing the at least one fluid to create an immunoglobulin package delivery system.

4. The method of claim 1 wherein the at least one producing animal is inoculated with more than one target immunogen.

5. The method of claim 1 wherein the at least one producing animal is selected from the group consisting of cows, horses, sheep, camels, goats, rabbits and llamas.

6. The method of claim 3 wherein the delivery system is selected from the group consisting of powder, capsules, spray, gel, liquid, salve, ointment, or cream.

7. The method of claim 1 wherein the processing step further comprises mixing the immunoglobulin package with a carrier material.

8. The method of claim 7 wherein the carrier material is selected from the group consisting of soybean oil, molasses, whey, rice hulls, soy bean hulls, pellets, distilled dried grains, boluses, tablets, PBS buffer, vitamin E solution and beet pulp.

9. The method of claim 1 wherein the at least one fluid harvested from the producing animal is plasma.

10. The method of claim 9 wherein the producing animal is a pregnant animal and the inoculation step occurs in the third trimester of the pregnancy.

11. The method of claim 10 wherein the at least one fluid harvested from the producing animal is colostrums.

12. The method of claim 11 wherein the at least one fluid harvested from the producing animal is plasma and colostrums.

13. The method of claim 12 wherein the harvesting step further comprises:
waiting until the producing animal has given birth;
collecting colostrums from the first four milkings of the producing animal after birth;
storing the collected colostrums;
waiting a predetermined period of time;
collecting plasma from the producing animal at predetermined intervals;
storing the plasma;
testing the plasma for immunoglobulin levels; and
continuing to collect, store and test plasma from the producing animal until the immunoglobulin level drops below a predetermined level.

14. The method of claim 1 wherein the producing animal is a bovine and the selecting step further comprises testing the producing animal for Bovine Viral Diarrhea, Johne's disease and mycoplasma infection.

15. The method of claim 1 wherein the producing animal is an equine and the selecting step further comprises testing the producing animal for Equine infectious anemia, Piroplasmosis, Dourine, Glanders, Brucellosis, and Influenza.

16. The method of claim 1 wherein the at least one producing animal further comprises a first producing animal and a second producing animal.

17. The method of claim 16 further comprising:
selecting a first and a second producing animal;
selecting a first and a second target immunogen;
inoculating the first producing animal with the first target immunogen and inoculating the second producing animal with the second target immunogen;
harvesting a first fluid containing immunoglobulins the first producing animal and a second fluid containing immunoglobulins from the second producing animal;
processing the first fluid containing immunoglobulins to create a first immunoglobulin package and the second fluid containing immunoglobulins to create a second immunoglobulin package; and
combining the first immunoglobulin package and the second immunoglobulin package to create a broad spectrum immunoglobulin package.

18. The method of claim 1 wherein at least one target immunogen is selected from the group consisting of Haemophilus, Rhinoviruses, Adenovirus, H1 Influenza, H3 Influenza, H5 Influenza, Mycoplasma, Pseudomonas, Aeromonas and Bordetella.

19. The method of claim 1 wherein at least one target immunogen is selected from the group consisting of Neisseria, Streptococcus, Staphylococcus, Chlamydia, Corynebacterium, Propionibacterium, Mycobacterium and Candidia.

20. The method of claim 1 wherein at least one target immunogen is selected from the group consisting of Bovine respiratory syncytial virus, Bovine viral diarrhea, adenoviruses, bovine parainfluenza 3, and infection bovine rhinotracheitis virus.

21. The method of claim 1 wherein at least one target immunogen is selected from the group consisting of Eastern Encephalomyelitis, Western Encephalomyelitis, Venezuelan Encephalomyelitis, Influenza A1, Influenza A2, EHV1 and EHV4.

22. The method of claim 1 wherein at least one target immunogen is a Gram-positive microbe.

23. The method of claim 22 wherein at least one target immunogen is selected from the group consisting of Streptococcus, Staphylococcus, Bacillus, Listeria, Clostridium, botulium, tetanus, Corynebacterium, methicillin resistant Straphylococcus aureus and Enterococcus

24. The method of claim 1 wherein at least one target immunogen is a Gram-negative microbe.

25. The method of claim 24 wherein at least one target immunogen is selected from the group consisting of E. coli, Salmonella, Haemophilus, Helicobacter, Campylobacter, Eschericha, Klebsiella, Shigella, Vibro, Cryptosporidium, Coccoidia and Yersinia.

26. The method of claim 1 wherein at least one target immunogen is selected from the group consisting of Enterococcus, E. coli, Endotoxin, Pseudomonas, Enterovirus, Clostridium perfringens toxin type A, Clostridium perfringens toxin type B, Clostridium perfringens toxin type C, Clostridium perfringens toxin type D, Clostridium perfringens toxin type E, Clostridium difficile, Rotavirus, Coronavirus, Giardia, Cryptospiridium, Toxoplasma, Salmonella, Shigella, Vibrio, Actinomyces, Campylobacter, Eubacterium, and Helicobacter.

27. The method of claim 1 wherein at least one target immunogen is selected from the group consisting of P. multicoda, M. haemolytica, H. somnus and H. suis.

28. The method of claim 1 wherein at least one target immunogen is selected from the group consisting of Mycoplasma pleuropneunmoniae, M. hypopneumoniae and M. bovis.

29. The method of claim 1 wherein at least one target immunogen is an influenza virus.

30. The method of claim 1 wherein at least one target immunogen is Porcine Reproductive and Respiratory Syndrome virus.
